(19) United States
(12) Patent Application Publication
(10) Pub. No.: US 2022/0074950 A1
(43) Pub. Date: Mar. 10, 2022

(54) METHOD FOR DETECTING HOST CELL PROTEINS IN THERAPEUTIC ANTIBODIES BY COMBINING TRYPSIN DIGESTION, CHROMATOGRAPHY GRADIENTS, AND BOXCAR MASS SPECTROMETRY

(71) Applicant: Regeneron Pharmaceuticals, Inc., Tarrytown, NY (US)

(72) Inventors: Song Nie, Armonk, NY (US); Tyler Greer, Elmsford, NY (US); Reid O'Brien Johnson, Hartsdale, NY (US); Xiaojing Zheng, Croton on Hudson, NY (US); Ning Li, New Canaan, CT (US)

(21) Appl. No.: 17/469,308

(22) Filed: Sep. 8, 2021

Related U.S. Application Data
(60) Provisional application No. 63/075,617, filed on Sep. 8, 2020.

(51) Int. Cl. G01N 33/68; C07K 14/71

(52) U.S. Cl. CPC G01N 33/6848; C07K 2319/30; C07K 14/71

(57) ABSTRACT

The present disclosure provides an improved method for profiling the nature of undesirable host cell proteins (HCPs) in a therapeutic antibody preparation using an improved assay. The assay includes three (3) exemplary steps comprising: ultra-low trypsin digestion, long gradient liquid chromatography, and mass spectrometry (MS) using, in particular, BoxCar mass spectrometry. The disclosure allows for determining the purity of a therapeutic antibody such that it is suitable for use in patients.

### CROSS-REFERENCE TO RELATED APPLICATIONS

This application claims benefit of U.S. Provisional No. 63/075,617 filed Sep. 8, 2020, which is herein incorporated by reference in its entirety.

### TECHNICAL FIELD

The present disclosure relates generally to methods for profiling the nature of host cell proteins (HCPs) in a therapeutic antibody preparation using an improved assay. The assay includes three (3) exemplary steps comprising: ultra-low trypsin digestion, long gradient liquid chromatography, and mass spectrometry (MS), in particular, BoxCar mass spectrometry.

### BACKGROUND

Therapeutic antibodies have revolutionized medicine and comprise a significant fraction of recently developed drugs. Substantial investment into their development has resulted in the approval of 79 therapeutic monoclonal antibodies (mAbs) by the US Food and Drug Administration, and revenue generated by the global mAb market is projected to reach $300 billion by 2025.

The increasing adoption of mAb therapies to a host of disorders is predominantly due to their high specificity, affinity to many drug targets, and minimal side effects. However, these boons are moderated by a mAb's prodigious size and chemical heterogeneity, as well as the presence of host cell protein (HCP) contaminants produced during cell expression in conjunction with the therapeutic protein.

The HCP population of a therapeutic is of utmost concern as it may contain proteins that risk patient safety or reduce drug efficacy. For example, immunogenic HCPs can elicit an unintended and harmful immune response in patients, while HCPs with enzymatic activity can degrade the therapeutic antibody itself or react with a component in the formulation buffer to decrease antibody stability and increase visible particulate formation.

Indeed, there are even reported cases of HCPs which are both immunogenic and have enzymatic potential. These risks necessitated the classification of HCP levels as a critical quality attribute (CQA) that must be monitored and controlled to acceptable levels. Regulatory guidelines do not provide a specific limit for HCP abundances, but common practice employs a risk-based approach with a maximum total value of 100 ppm or less in the purified drug product.

Decreasing HCP abundances to such low levels requires implementing a multi-step chromatographic process capable of virtually eliminating thousands of proteins with substantial ranges in size, net charge, hydrophobicity, and many other physicochemical properties. Such processes typically begin with Protein A affinity chromatography, which removes most HCPs, followed by orthogonal purification steps like anion exchange, size exclusion, or hydrophobic interaction chromatography to further reduce the population and abundance of lingering contaminants. (Gilgunn, S. et al. Bones, J. J Chromatogr A 2019, 1595, 28-38; Liu, H. F. et al. MAbs 2010, 2, 480-499; Zhu, M. M. et al. In Handbook of Industrial Chemistry and Biotechnology, 2017, pp 1639-1669.)

Since a successful therapeutic antibody purification strategy reduces total HCP levels below 100 ppm, monitoring these low abundance analytes is challenging and often requires multiple assays. HCP detection and quantification strategies are commonly initiated with enzyme-linked immunosorbent assays (ELISA). This popularity results from the method's high specificity, accuracy, and precision, as well as its ease of use and automation. (Zhu-Shimoni, J. et al. Biotechnol Bioeng 2014, 111, 2367-2379; Rey, G. et al. J Pharm Biomed Anal 2012, 70, 580-586.)

However, ELISA's advantages are accompanied by several limitations that reduce its utility. Of particular importance, ELISA requires polyclonal antibodies produced by immunizing animals with a null cell line, which does not fully cover all HCPs from the production cell line and results in biased detection of more immunogenic HCPs. (Zhu-Shimoni, J. et al. M. Biotechnol Bioeng 2014, 111, 2367-2379; Henry, S. M. et al. MAbs 2017, 9, 1065-1075.) Underestimation of HCP levels is also common in cases where proteins are noncovalently bound to the therapeutic antibody or their concentrations are higher than the capacity of the polyclonal antibodies. (Anicetti, V. R. et al. J Immunol Methods 1986, 91, 213-224.)

Finally, while ELISA kits that detect a single, important HCP (like phospholipase B-like 2) exist and can be developed for other known, problematic proteins, conventional ELISA platforms measure only the total protein concentration and cannot identify specific proteins or quantify their individual concentrations. (Henry, S. M. et al. MAbs 2017, 9, 1065-1075.) These limitations create unacceptable gaps in HCP characterization that regulatory agencies now expect to be filled through orthogonal methods like liquid chromatography-tandem mass spectrometry (LC-MS2).

For these reasons, it is beneficial to have robust and highly accurate assays using analytical physical chemistry for high speed and accurate HCP quality control (deep profiling) of protein samples, for example, therapeutic antibodies. It is desirable for such assays to have wide application for perfecting the manufacture of antibodies in clinical development and in commercial use, higher overall HCP control as compared to published and established methods, higher identity coverage and fidelity as sensitive as about 0.1 ppm, and higher speed, ease of use (minimal steps), and lower cost as compared to other methods.

### SUMMARY

In one aspect, the present disclosure relates to a method of resolving unacceptable gaps in HCP characterization with improved speed and accuracy. In particular, the present disclosure provides high speed assays for determining the identity or amount of a contaminating protein in a therapeutic protein sample comprising typically three (3) steps comprising subjecting a therapeutic protein sample to: 1) ultra-low trypsin digestion, 2) long gradient liquid chromatography, and 3) mass spectrometry (MS), in particular, BoxCar mass spectrometry. The above three steps, i.e., Ultra-Low Trypsin concentration digestion, Long gradient liquid chromatography, and BoxCar mass spectrometry, are referred to herein by the acronym ULTLB.

In one embodiment, an assay may be suitable for determining in parallel the identity and abundance of a contaminating protein, in particular, a host cell protein (HCP).

In one embodiment, the assay may determine the identity of a contaminating protein by amino acid sequence or partial sequence, sufficient to identify the protein.

In some embodiments, the assay may determine the abundance of a contaminating protein at levels measured in parts per million (ppm), wherein the amount of the contaminating protein is determined with a sensitivity in parts per million (ppm) at a level as low as about 10, 5, 2, 1, 0.1 or less and intervals thereof, i.e., less than 10 ppm, less than 5 ppm, less than 2 ppm, less than 1 ppm or less than 0.1 ppm.

In embodiments, the assay, in principle, may be used to interrogate any therapeutic protein sample for the presence of HCP impurities and in particular, for therapeutic proteins such as an antibody, antibody variant, or antibody fusion.

Accordingly, the assay may be used for applying to the purification stream of any number of therapeutic antibodies, variants, and fusions listed herein (see subsection entitled “Wide Application of the Assay”).

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1: shows a schematic of an illustrative assay having three (3) key steps as indicated: Step 1 shows a therapeutic antibody sample having host cell proteins (HCPs) being subjected to an ultra-low trypsin concentration digestion process; Step 2 shows the resultant digested polypeptides being subjected to a long gradient liquid chromatography process; and Step 3 shows the polypeptides of previous Step 2 being subjected to a BoxCar Mass Spectrometry Acquisition process resulting (as shown in the stacked Venn diagram) a significant improvement as compared to previous methods.

The above three steps, i.e., Ultra-Low Trypsin concentration digestion, Long gradient liquid chromatography, and BoxCar mass spectrometry, is referred to herein by the acronym ULTLB, as indicated in FIG. 1. The term “BoxCar” refers to, e.g., a selective MS scan comprised of 12 isolation windows, i.e., “boxes” to achieve greater fidelity (see also FIG. 3).

FIG. 2A: shows antibody samples comprising an antibody standard (NISTmAb) when subjected to varying trypsin digestion conditions of decreasing strength, going left to right, from Normal, Native, and Ultra-Low. Each sample was run in duplicate.

The left panel shows the highest amount of HCPs are identified by using Ultra-Low conditions as compared to Normal and Native digestion conditions.

The right panel shows the highest amount of unique peptides are identified by using Ultra-Low conditions, as compared to Normal and Native digestion conditions.

FIG. 2B: shows antibody samples comprising the antibody standard (NISTmAb) of the above-mentioned digestion conditions when subjected to varying liquid chromatography (LC) column length (25 cm or 50 cm) and gradient length (2 hrs or 4 hrs), going left to right. Each sample was run in duplicate.

The left panel shows the highest amount of HCPs are identified by using long column length (50 cm) and long gradient (4 hrs) as compared to short column length (25 cm) and short gradient (2 hrs).

The right panel shows the highest amount of unique peptides identified by using long column length (50 cm) and long gradient (4 hrs) as compared to short column length (25 cm) and short gradient (2 hrs).

FIGS. 3A-3C: show BoxCar acquisition profiles that improved HCP identification by boosting the MS signal of low abundance peptides. FIG. 3A shows a representative example of a standard full scan. FIG. 3B shows the adjacent BoxCar scan composed of 12 narrow isolation windows (‘boxes’). In FIG. 3A and FIG. 3B, the shaded insets highlight an observable peptide signal increase in the BoxCar scan compared to the full scan. Signal-to-noise ratios (S/N) are provided for comparison. FIG. 3C shows a significant improvement in HCP identifications (about 51%) were obtained by BoxCar acquisition samples compared to traditional Data Dependent Acquisition (DDA).

FIG. 4: shows in Panel A the contribution to the overall improvement of HCP identifications using the ULTLB method from the optimization of sample preparation, LC separation, and MS acquisition. Figure legends are as follows: Native: native digestion; 25 cm and 50 cm indicate column length; 2 h and 4 h represents gradient time. In Panel B, a stacked Venn diagram of the total HCPs identified by ULTLB as compared to native digestion and MWCO enrichment methods is shown. In total, 453 HCPs were identified by methods including ULTLB.

FIG. 5: shows in Panel A, a stacked Venn diagram indicating highly consistent overlap (92%) of HCPs identified across duplicate runs (Rep 1 and Rep 2, as indicated). In Panel B, a Pearson Correlation is shown indicating high concordance of identified protein abundance across the duplicate runs (Rep 1 and Rep 2, as indicated).

FIG. 6: shows in Panel A, a stacked Venn diagram comparing two (2) previous methods of HCP detection for an exemplary therapeutic antibody REGN mAb1 (Protein A and Native Digestion) as compared with illustrative methods in accordance with illustrative embodiments described herein (methods using ULTLB). This information is also presented in tabular form. In Panel B, a stacked Venn diagram is shown comparing two (2) previous methods of HCP detection for a second exemplary therapeutic antibody REGN mAb2 (Protein A and Native Digestion) as compared to the illustrative methods in accordance with illustrative embodiments described herein (methods that utilize ULTLB). This information is also presented in tabular form.

### DETAILED DESCRIPTION

### 1. Definitions

The term “analytical technique or analytical chemistry” refers to the quantitative analysis of polypeptide molecules for the purpose of carrying out illustrative methods, using, for example, liquid chromatography (LC), mass spectrometry (MS), or a combination thereof.

The term “antibody” refers to a therapeutic immunobinder, e.g., a monoclonal antibody, bi- or multi-specific antibody, that is suitable for introducing into a subject for modulating a disease or disorder, for example, an immune or oncological disorder. The term “antibody” is to be construed broadly as describing monoclonal antibodies, bispecific antibodies, antibody compositions with multi-specificity, as well as antibody fragments or subunits (e.g., Fab, F(ab′)2, scFv, Fv, Fd, Fc/2, and LC), antibody derivatives, fusions, variants, and analogs.

The term “BoxCar” refers to the analytical technique, for characterizing a polypeptide, known as an MS technique, wherein there are one or more windows or “boxes” that are selectively analyzed.

The term “DDA” refers to the analytical technique, for characterizing a polypeptide, known as Data Dependent Acquisition in connection with performing a Mass Spectrometry step.

The term “deep profiling” refers to the ability, using analytical techniques (e.g., MS), to achieve high output protein quantitation from a sample.

The terms “HCP” and “HCPs” refer to the singular and plural of a host cell protein(s) which are an undesirable impurity found in protein preparations when manufactured (i.e., genetically expressed) in host cells, typically eukaryotic cells, such as CHO cells.

The term “HRAM” refers to the analytical technique, for characterizing a polypeptide, known as high-resolution accurate-mass spectrometry.

The term “LC” refers to the analytical technique, for characterizing a polypeptide, known as liquid chromatography.

The term “LC-MS” refers to the analytical technique, for characterizing a polypeptide, known as liquid chromatography and mass spectrometry.

The term “LCMS2 refers to the analytical technique, for characterizing a polypeptide, known as liquid chromatography and tandem mass spectrometry.

The term “long gradient LC” refers to the analytical technique, for characterizing a polypeptide, known as liquid chromatography, wherein the long gradient refers to column size and residence time, e.g., 50 cm and 4 hrs, respectively.

The term “mAb” refers to a monoclonal antibody.

The term “MS” refers to the analytical technique, for characterizing a polypeptide, known as mass spectrometry.

The term “m/z” refers to an analytical parameter, for characterizing aspects of a polypeptide using, e.g., MS, wherein “m” stands for mass and “z” stands for the charge number of ions observed.

The term “NISTmAb” refers to the monoclonal antibody standard that is known as the “National Institute of Standards & Technology Humanized IgG1κ Monoclonal Antibody standard (NISTmAb)”.

The term “Orbitrap” and “Exploris” refers to commercially available mass spectrometers well known in the field of analytical chemistry for characterizing polypeptides.

The term “trypsin digest or digestion” refers to a protein that has been exposed to a trypsin enzyme mediated cleavage step. The digest can be highly modified by trypsin concentration and incubation time to achieve desired results. In embodiments, the trypsin digest may be an “ultra-low digest” for example at a ratio of 10,000:1.

The term “polypeptide digest or peptide digest” refers to a polypeptide or peptide mix resultant from exposing a polypeptide, e.g., an antibody, as described herein, when incubated with one or more enzymes (e.g., trypsin) capable of digesting a larger protein or polypeptide sequence such that polypeptides or peptides of appropriate size can be interrogated using illustrative methods including ULTLB.

The term “ULTLB” is an acronym for the three-step assay referred to as the Ultra-Low Trypsin concentration digestion, Long gradient liquid chromatography, and BoxCar mass spectrometry.

The term “ultra-low trypsin concentration digestion” refers to a low trypsin ratio for achieving desirable peptide profiles.

Unless defined otherwise, all terms and phrases used herein include the meanings that the terms and phrases have attained in the art, unless the contrary is clearly indicated or clearly apparent from the context in which the term or phrase is used.

### 2. Improved Assays for Characterizing the Nature and Abundance of HCPs in a Therapeutic Antibody Preparation

The disclosure provides an assay typically comprising three steps:

Step 1 comprises subjecting a therapeutic antibody sample having host cell proteins (HCPs) to an ultra-low trypsin concentration digestion process;

Step 2 comprises subjecting the resultant digested polypeptides to a long gradient liquid chromatography process; and

Step 3 comprises subjecting the polypeptides of previous step 2 to a BoxCar Mass Spectrometry Acquisition process resulting in a significant improvement in the number of polypeptides identified both in kind and in abundance.

Illustrative methods, and each step, are described in multiple examples below. Importantly, illustrative assays in accordance with this disclosure may be applied to several therapeutic antibodies with superior results as compared to known techniques.

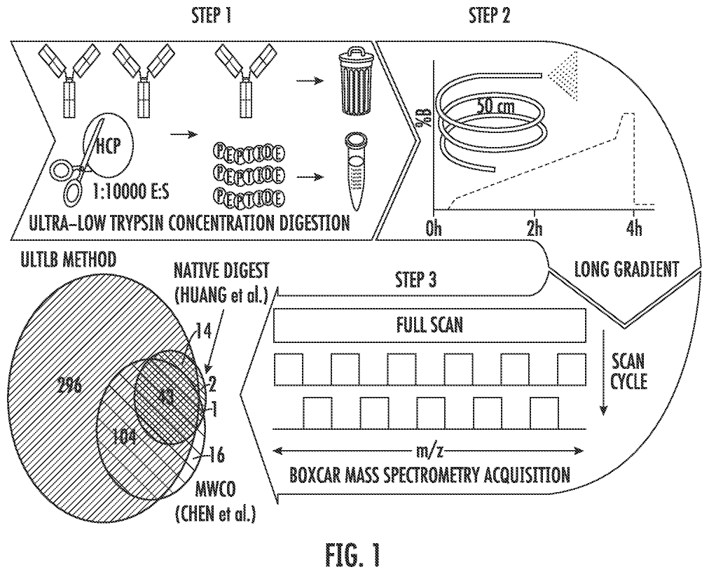

The above three steps, i.e., Ultra-Low Trypsin concentration digestion, Long gradient liquid chromatography, and BoxCar mass spectrometry, is referred to herein by the acronym ULTLB and presented in schematic form in FIG. 1.

Illustrative assays described herein utilize parameters that achieves high fidelity with speed and ease of use. Illustrative assays further allow for antibody therapeutics to be screened of any HCP impurities in order to safeguard patients.

### 3. Wide Application

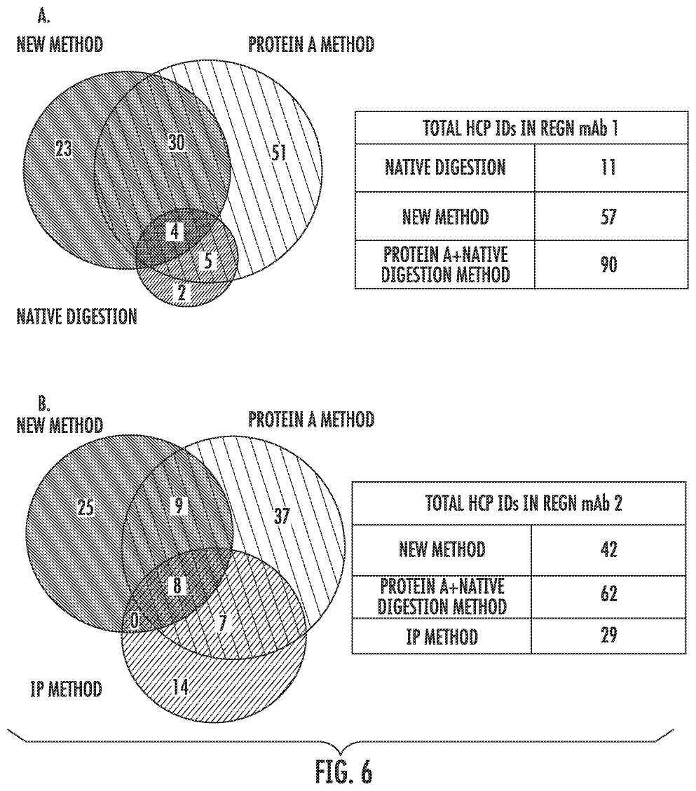

It should be appreciated that the present disclosure provides for the fast and accurate determination of the identity and abundance of host cell proteins (HCPs) in a therapeutic antibody preparation with high fidelity (within about 0.1 ppm). Illustrative assays were also been applied to two (2) exemplary therapeutic antibodies (REGN mAb1 and REGN mAb2) with superior results (See FIG. 6. upper panel and lower panel, respectively). Accordingly, the disclosed methods compliment and improve the CMC (Chemistry, Manufacturing, and Controls) of any commercially produced therapeutic antibody.

For example, in one aspect, illustrative assays allow for perfecting the manufacture and safeguarding of the homogeneity and purity of a number of antibody therapies.

Such antibody therapies include: abciximab, adalimumab, adalimumab-adbm, adalimumab-atto, ado-trastuzumab emtansine, alemtuzumab, alirocumab, atezolizumab, avelumab, basiliximab, belimumab, benralizumab, bevacizumab, bevacizumab-awwb, bezlotoxumab, blinatumomab, brentuximab vedotin, brodalumab, burosumab-twza, canakinumab, capromab pendetide, certolizumab pegol, cetuximab, daclizumab (Zenapax®), daclizumab (Zinbryta®), daratumumab, denosumab, dinutuximab, dupilumab, durvalumab, eculizumab, elotuzumab, emicizumab-kxwh, erenumab-aooe, evolocumab, gemtuzumab ozogamicin, golimumab, guselkumab, ibalizumab-uiyk, ibritumomab tiuxetan, idarucizumab, infliximab, infliximab-abda, infliximab-dyyb, infliximab-qbtx, inotuzumab ozogamicin, ipilimumab ixekizumab, mepolizumab, natalizumab, necitumumab, nivolumab, obiltoxaximab, obinutuzumab, ocrelizumab, ofatumumab, olaratumab, omalizumab, palivizumab, panitumumab, embrolizumab, pertuzumab, ramucirumab, ranibizumab, raxibacumab, reslizumab, rituximab, sarilumab, secukinumab, siltuximab, tildrakizumab-asmn, tocilizumab, trastuzumab, trastuzumab-dkst, ustekinumab, vedolizumab, and rituximab and hyaluronidase.

Other therapeutic antibodies of interest for various indications subject to illustrative assays include: aflibercept, for treating eye disorders; rilonacept for treating blindness and metastatic colorectal cancer; alirocumab for treating familial hypercholesterolemia or clinical atherosclerotic cardiovascular disease (ASCVD); dupilumab for treating atopic dermatitis; sarilumab for treating rheumatoid arthritis and COVID-19; cemiplimab for treating PD-1 related disease; and antibodies for treating Ebola.

### EXEMPLIFICATION

The examples below are provided for illustrative purposes and should not be construed as limiting the disclosure which is defined by the appended claims. All references and patents recited within the present application are included herein by reference.

### Materials and Methods

### REFERENCES

The methods of the present disclosure, when practiced by the person skilled in the art, may make use of conventional techniques in the field of physical chemistry for the analysis of peptides and proteins, are provided within the disclosure as well as described in the following references, and current electronic versions, such as “Introduction to Protein Mass Spectrometry” by Pradip Kumar Ghosh, 2015, “Analytical Characterization of Biotherapeutics” by Jennie R. Lill and Wendy Sandoval, 2017, “Mass Spectrometry of Proteins and Peptides: Methods and Protocols Second Edition (Methods in Molecular Biology)” by Mary S. Lipton and Ljiljana Paša-Tolic, 2008, “Advancements of Mass Spectrometry in Biomedical Research” by Alisa G. Woods and Costel C. Darie, 2019; and “Protein Analysis using Mass Spectrometry: Accelerating Protein Biotherapeutics from Lab to Patient” by Mike S. Lee and Qin C. Ji, 2017.

Materials

Trifluoroacetic acid (TFA), formic acid (FA), and acetonitrile were purchased from Thermo Fisher Scientific (Rockford, Ill.). Urea, iodoacetamide (IAM), tris(2-carboxyethyl) phosphine hydrochloride (TCEP-HCl) and humanized IgG1κ monoclonal antibody standard RM 8671 were obtained from Sigma-Aldrich (St. Louis, Mo.).

Sequencing grade modified trypsin with resuspension buffer was obtained from Promega (Madison, Wis.) and Tris-HCl buffer (pH 7.5) was obtained from Invitrogen (Carlsbad, Calif.). C18 SPE columns were obtained from Waters (Milford, Mass.). Purified monoclonal antibody and spiked-in CHO proteins were produced internally by Regeneron (Tarrytown, N.Y.).

Normal Digestion

Drug substance samples of 200 μg were diluted to 5 mg/ml using 9 M urea/100 mM Tris-HCl. Disulfide bonds were reduced with 10 mM DTT and incubated for 30 min at 50° C. Samples were cooled to room temperature and alkylated with 15 mM IAM for 30 min in the dark.

The reduced and alkylated samples were diluted roughly 8-fold using 100 mM Tris-HCl. Proteolytic digestion was performed with trypsin (1:20 trypsin:substrate ratio) overnight at 37° C. The digestion was quenched by acidifying to 0.2% FA. Samples were desalted by C18 SPE columns before nanoLC-MS/MS analysis.

Native Digestion

A detailed description of the native digestion is provided by Huang et al. 2017 (Anal Chem 2017, 89 5435-5444). Briefly, samples were diluted to about 5 mg/ml using 50 mM Tris-HCl buffer, pH 8. Proteins were then digested with trypsin (1:400 w/w enzyme:substrate ratio) at 37° C. overnight with a final pH of about 7.4.

Subsequently, disulfide bonds were reduced with 5 mM TCEP and incubated for 10 min at 90° C. The resulting peptide mixtures were acidified to about 0.2% FA and centrifuged at 15000 g for 2 min. Supernatant was transferred to 10 kDa Amicon molecular weight cutoff (MWCO) centrifugal filters to remove particles before nanoLC-MS/MS analysis.

Low Trypsin Concentration Digestion

Samples were desalted and buffer exchanged into 50 mM Tris-HCl buffer, pH 7.5 using 10 kDa Amicon MWCO centrifugal filters. The protein concentration was measured using a NanoDrop 2000 Spectrophotometer from Thermo Scientific and diluted to about 5 mg/ml. Then, trypsin diluted in resuspension buffer (50 μg/μ1), was added to samples at a 10000:1 w/w protein:trypsin ratio to digest at 37° C. overnight. Subsequently, disulfide bonds were reduced with 5 mM TCEP and incubated for 10 min at 90° C. Samples were acidified to about 0.2% FA and centrifuged at 15000 g for 2 min. Supernatant was transferred to 10 kDa MWCO filters to remove particles before nanoLC-MS/MS analysis.

NanoLC-MS/MS

All HCP samples were analyzed using an UltiMate 3000 RSLCnano system (Thermo Scientific) coupled to an Orbitrap Exploris 480 mass spectrometer (Thermo Scientific). The RSLCnano system was equipped with a 25 cm or 50 cm C18 column (CoAnn Technologies, 360 μm OD, 75 μm ID, 10 μm tip ID).

Mobile phase A contained 0.1% FA in water and mobile phase B contained 0.1% FA in 80% acetonitrile/20% water. Samples were loaded on an Acclaim PepMap 100, 75 μm×2 cm pre-column (Thermo Scientific) for 5 min at a flow rate 5 μl/min. For the 2 h gradient, a linear LC gradient was set up as follows: 5% B at 0 min, 8% B at 8 min, 36% B at 95 min, and 95% B from 115-120 min.

For the 4 h gradient, a linear LC gradient was set up as follows: 5% B at 0 min, 8% B at 10 min, 36% B at 220 min, and 95% B from 235-240 min. The flow rate was 0.25 μl/min for the 25 cm column and 0.2 μl/min for the 50 cm column.

Mass spectra data acquisition was performed using Xcalibur v4.3 (Thermo Fisher Scientific, CA). The nano ESI spray voltage was set at 2200 V. In traditional data dependent acquisition mode (DDA), a survey scan was performed in the Orbitrap with a cycle time of three seconds. MS full scans were acquired from m/z 380-1500 at 60K resolution (m/z 200) with 300% standard automated gain control (AGC), and a maximum injection time of 20 ms.

MS/MS fragmentation was performed using HCD with a normalized collision energy of 30% at a resolution of 15K (m/z 200), 75% standard AGC, and a maximum injection time of 50 ms. Dynamic exclusion duration was set to 45 seconds with a single repeat count, and only precursors with charge states of +2 to +6 were selected.

BoxCar Scan on Exploris 480

The BoxCar scan settings followed those published by Meier et al. Nat Methods 2018, 15, 440-448 with some modifications. The overall acquisition cycle comprised a standard full scan and then two BoxCar scans covering a range of m/z 400-1200 with a cycle time of 1.5 s. Suitable precursor ions were selected from the preceding full scan, applying the same criteria and settings as in the traditional DDA experiments.

Each BoxCar has 12 different boxes with different m/z windows. For each BoxCar scan, the total AGC target value was adjusted to 300% standard AGC and evenly distributed across all boxes. For each of the boxes, the maximum total ion injection time was 20 ms. All other parameters are identical to standard DDA as previously described.

Data Analysis

Database searches for HCP protein identification were performed using SEQUEST and Mascot embedded into Proteome Discoverer 2.2 (Thermo Fisher Scientific) against the SwissProt mouse target-decoy protein database, which included common contaminants. Precursor ion mass tolerance was set to 20 ppm and fragment ion mass tolerance was set to 0.02 Da.

Trypsin was specified as the digestion enzyme during the database search with one missed cleavage allowed. Methionine oxidation (+16 Da) was selected as a variable modification and cysteine carbamidomethylation was chosen as a fixed modification for the normal (alkylated) digests.

False discovery rates (FDRs) were set to 1% for peptide identification and 5% for protein identification, with a minimum of 2 unique peptides detected per protein.

### Example 1

### Design Aspects and Improvements of the HCP Assay for Interrogating Therapeutic Antibody Preparations for Purity

This example describes many of the challenges and design aspect solutions considered in building illustrative assays for detecting HCPs in therapeutic antibody preparations.

The coupling of highly resolved LC separations and sensitive, high-resolution accurate-mass (HRAM) mass spectrometers enables identification and quantification of individual HCPs independent of their immunogenicity. The chief obstacle in such analyses arises from the vast concentration difference between the abundant therapeutic antibody and individual, low abundance HCPs (less than 1-100 ng HCP per mg therapeutic).

As purification strategies for therapeutic antibodies have advanced, the dynamic range between the therapeutic and contaminant proteins has only increased, and most low abundance HCPs, which may still have immunogenic or enzymatic activity, cannot be detected by direct LC-MS2 methods alone.

In response to this challenge, a multitude of sample preparation and instrumental techniques have been developed that: 1) lessen the concentration gulf between HCPs and the therapeutic protein through immunoaffinity, protein A, or molecular weight cutoff, 2) reduce overlap between HCP and therapeutic peptide peaks using multi-dimensional chromatography, and 3) circumvent intra-scan dynamic range restrictions that hamper data-dependent analysis of species falling outside of the lower limit. (Henry, S. M. et al. MAbs 2017, 9, 1065-1075; Thompson, J. H. et al. Rapid Commun Mass Spectrom 2014, 28, 855-860; Madsen, J. A. et al. MAbs 2015, 7, 1128-1137; Chen, I. H. et al. Anal Chem 2020, 92, 3751-3757; Doneanu, C. E. et al. MAbs 2012, 4, 24-44; Farrell, A. et al. Anal Chem 2015, 87, 9186-9193; Kufer, R.; Haindl, M. et al. Anal Chem 2019, 91, 9716-9723; Walker, D. E. et al. MAbs 2017, 9, 654-663; Doneanu, C. E. et al. Anal Chem 2015, 87, 10283-10291; Kreimer, S. et al. Anal Chem 2017, 89, 5294-5302; Johnson, R. O. et al. Anal Chem 2020; Wang, Q. et al. Anal Chem 2020.)

While some of these methods can detect down to sub-ppm level protein contaminants, many are preferentially selective towards certain HCPs, reduce throughput significantly, or increase the difficulty of identifying novel proteins. As such, relatively simple LC-MS2 methods that substantially increase depth of HCP coverage without introducing significant bias or other caveats are in high demand throughout the biopharmaceutical industry and regulatory agencies.

Liquid chromatography coupled to mass spectrometry (LC-MS) is a powerful tool for the analysis of host cell proteins (HCP) during antibody drug process development due to its sensitivity, selectivity, and adaptability. However, the enormous dynamic range between the therapeutic antibody and accompanying HCPs poses a significant challenge for LC-MS based detection of these low abundance impurities.

To address this challenge, enrichment of HCPs via immunoaffinity, protein A, 2D-LC, or other strategies is typically performed. However, these enrichments are time-consuming and sometimes require a large quantity of sample.

Disclosed herein is a simple and sensitive strategy to analyze HCPs in therapeutic antibody samples without cumbersome enrichment by combining an ultra-low trypsin concentration during digestion under non-denaturing conditions, a long chromatographic gradient, and BoxCar acquisition (ULTLB) using a quadrupole-Orbitrap mass spectrometer.

Application of this strategy to the NIST monoclonal antibody standard (NISTmAb) resulted in the identification of 453 mouse HCPs, which is a significant increase in the number of identified HCPs without enrichment compared to previous reports. Known amounts of HCPs were spiked into the purified antibody drug substance, demonstrating that the method sensitivity is as low as 0.1 ppm. Thus, the ULTLB method represents a sensitive and simple platform for deep profiling of HCPs in antibodies.

Combining advancements of sample preparation, LC separation and MS data collection likely hold the key to developing methods capable of deep HCP profiling without the usual encumbrances. One such development preferentially digests HCPs over the antibody by utilizing non-denaturing conditions. (Huang, L. et al. Anal Chem 2017, 89, 5436-5444). Another proteomics study, although not applied to HCPs, observed enhanced detection of low abundance proteins by applying an extremely low trypsin concentration (1:25000 w/w enzyme:substrate ratio) during digestion (Fonslow, B. R. et al. Nat Methods 2013, 10, 54-56).

Proteomics investigations regarding LC column and gradient lengths have shown that longer columns and gradients result in sensitive and deep proteome coverage without additional sample preparation and the burdensome time requirements of multi-dimensional LC (Thakur, S. S. et al. Mol Cell Proteomics 2011, 10, M110 003699; Hinzke, T. et al. Front Microbiol 2019, 10, 238).

Lastly, a novel acquisition method for Orbitrap mass spectrometers, coined the “BoxCar” method, reduces intra-scan dynamic range issues plaguing full scan MS1 acquisition during DDA by filling the C-trap with ions from narrow m/z windows and sequentially transmitting these packets to the Orbitrap, where they are analyzed in a single scan (Meier, F. et al. Nat Methods 2018, 15, 440-448). The BoxCar technique allows lower abundance ions more acquisition time and can increase the signal to noise by a full order of magnitude by restricting the space that high abundance ions consume in the trap.

In this study, an examination of multiple columns and gradient lengths revealed that longer lengths for both maximize HCP identifications, and BoxCar acquisition was found to identify more HCPs than conventional DDA. Furthermore, by combining an ultra-low trypsin concentration during digestion under non-denaturing conditions with a long chromatographic gradient and BoxCar mass spectrometry acquisition on a quadrupole-Orbitrap mass spectrometer, a simple and novel platform for deep profiling of HCPs was established. Termed the “ULTLB” method as mentioned herein, this simple, robust, and fast protocol enables much deeper profiling of HCPs compared to other methods because the ULTLB method obviates unnecessary additional time-consuming sample enrichment steps.

As disclosed herein, an examination of multiple columns and gradient lengths revealed that longer lengths for both maximize HCP identifications, and BoxCar acquisition was found to identify more HCPs than conventional DDA. Furthermore, by combining an ultra-low trypsin concentration during digestion under non-denaturing conditions with a long chromatographic gradient and BoxCar mass spectrometry acquisition on a quadrupole-Orbitrap mass spectrometer, it was established that a simple and novel platform for deep profiling of HCPs, was needed.

Termed the “ULTLB” method, this simple, robust, and fast protocol enables much deeper profiling of HCPs compared to other methods and obviates unnecessary additional time-consuming sample enrichment steps (See FIG. 1, Example 6, and Table 2). As shown in FIG. 1, the assay of the present disclosure identified 296 polypeptides. Chen et al. identified only 104 polypeptides. Huang et al. identified only 43 polypeptides.

Accordingly, this example sets out the challenges of HCP detection and sets forth various parameters of illustrative methods described herein (methods using ULTLB) that significantly improve HCP detection, as further described in detail below.

### Example 2

### Assay Design and Results Pertaining to Step 1 of the 3 Step Assay: The Trypsin Digestion Step

This example describes design aspects of illustrative assays for detecting HCPs in therapeutic antibody preparations beginning with the first of three steps of the assay: The Trypsin Digestion Step.

A simple and sensitive sample preparation method without enrichment steps was developed using a novel digestion strategy. This digestion step involved using an ultra-low concentration of trypsin in non-denaturing conditions which resulted in the preferential digestion of HCPs over the mAb itself.

The mAb, typically being much larger than most HCPs and stabilized by a total of 16 disulfide bonds, is less accessible than HCPs to trypsin and is readily digested under reducing conditions or with relatively high levels of protease. To obtain the optimum digestion conditions, different protein:trypsin ratios from 25000:1 to 1000:1 were tested by digesting NISTmAb in non-denaturing conditions.

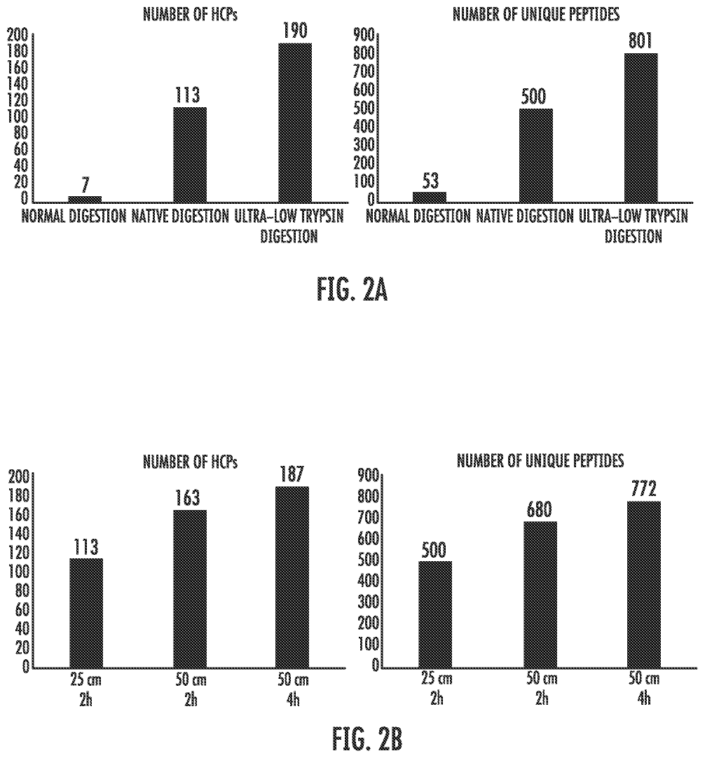

All samples were analyzed on a 25 cm column with a 2 h gradient. FIG. 2 shows that more HCPs and unique peptides were identified from digests with protein:trypsin ratios of 10000:1 and 2500:1 compared to digests with protein:trypsin ratios of 25000:1 and 1000:1. Further analysis revealed that the number of identified mAb peptide spectra increased multi-fold with increasing trypsin concentration (about 1000 vs about 4200).

Relatively few mAb spectra were identified in the ultra-low trypsin concentration digestion samples (25000:1 and 10000:1), which indicates a minimal amount of mAb was digested in these samples. By leveraging the number of HCPs identified against how many mAb peptide spectra were found, the protein:trypsin ratio of 10000:1 was selected as the optimum protein:trypsin ratio, where the most HCPs were detected and a minimum amount of mAb was digested.

To compare this method to normal and native digestions, samples prepared under each condition were analyzed using the same LC-MS conditions. As shown in FIG. 2, the ultra-low concentration trypsin digestion method improves HCP identifications significantly (by 68% at the protein level and 60% at the peptide level) compared to the native digestion method. Both methods performed substantially better than the normal digestion condition, which detected only a few HCPs and peptides.

The main reason for reduced HCP detection is the high abundance of digested antibody peptides which dominated the LC chromatogram in the normal digest samples and are still highly abundant in the native digestion samples. However, the ultra-low trypsin concentration digestion samples have a low number of mAb digested peptides, and therefore, less interference of HCP peptides.

The results demonstrate that the disclosed ultra-low concentration trypsin digestion is a simple method to selectively digest low abundance HCP peptides by keeping the mAb intact during sample preparation without any additional and tedious enrichment procedures. See FIGS. 2A and 2B.

Accordingly, this example sets out the challenges of HCP detection and sets forth various parameters of illustrative assays described herein (assays using ULTLB) that significantly improve the first of three steps of the assay: The Trypsin Digestion Step.

### Example 3

### Assay Design and Results Pertaining to Step 2 of the 3 Step Assay: The Long Gradient Liquid Chromatography Step

This example describes design aspects of illustrative assays for detecting HCPs in therapeutic antibody preparations beginning with the second of three steps of the assay: The Long Gradient Liquid Chromatography Step.

To optimize LC separation and improve HCP identification, a series of columns with different lengths and dimensions from multiple companies were tested to obtain the best separation using NISTmAb HCP samples (data not shown). The best performing columns (CoAnn Technologies, LLC, 1.7 μm, C18 column) were subsequently used for further column length and gradient optimizations using NISTmAb native digestion samples. Data from column length optimization experiments reveal that using the 50 cm column results in 44.2% more HCPs and 36.0% more unique peptide identifications compared to the 25 cm column when a 2 h gradient is used (FIG. 2).

The demonstrated superiority of the 50 cm column length is due to greater separation and narrower peak widths. Gradient length was further optimized on the 50 cm column. We found that HCP identification can be improved further by 14.7% at the protein level and 13.5% at the peptide level using a 4 h gradient compared to a 2 h gradient. However, even longer gradients (6 h and 8 h) were tested on the same column and no further improvement was observed. While proteomics studies have shown that 8 h gradients performed best when compared with 4 h or 6 h gradients in complex tissue or cell samples, 35,36 the advantages of using gradients of similar length appear to be diminished when analyzing HCP samples as they are less complex and have an unusually high concentration of a single protein (mAb) which interferes with low abundance HCP peptide analysis.

Furthermore, the MS signal of low abundance HCP peptides can decrease significantly due to widening peak widths in longer gradients like 6 or 8 h. These observations led us to choose a 4 h gradient on a 50 cm column as the optimum LC separation condition. About 65.5% more HCPs (187 vs 113) and 54.4% more unique peptides (772 vs 500) were identified in the NISTmAb native digestion samples when using a 4 h gradient and 50 cm column in lieu of the shorter 2 h separation on a 25 cm column (FIG. 2). Combining this optimum LC separation with our ultra-low trypsin concentration digestion results in more than three times the number of HCPs identified compared to the native digestion using the standard LC conditions (366 vs 113). See FIGS. 2A and 2B.

Accordingly, this example sets out the challenges of HCP detection and sets forth various parameters of illustrative assays described herein (assays using ULTLB) that significantly improve the second of three steps of the assay: The Long Gradient Liquid Chromatography Step.

### Example 4

### Assay Design and Results Pertaining to Step 3 of the 3 Step Assay: The Liquid Chromatography Mass Spectrometry (LC-MS) BoxCar Step

This example describes design aspects of illustrative assays for detecting HCPs in therapeutic antibody preparations beginning with the third of three steps of the assay: The Liquid Chromatography Mass Spectrometry (LC-MS) BoxCar Step

A major challenge for LC-MS based methods is that there can be more than six orders of magnitude in the concentration difference between HCPs and the therapeutic antibody in a sample digest. Furthermore, a key limitation of trapping-based mass spectrometers is the limited charge capacity of the ion trap, which excludes many low abundance ions from MS1-level analysis. To alleviate the wide dynamic range issue hampering HCP analysis, the BoxCar acquisition method was adapted to HCP sample analysis to increase detection dynamic range.

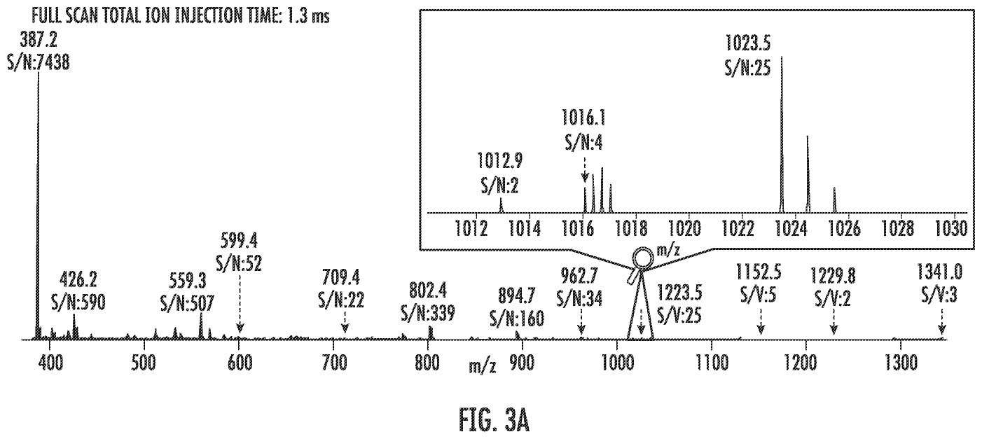

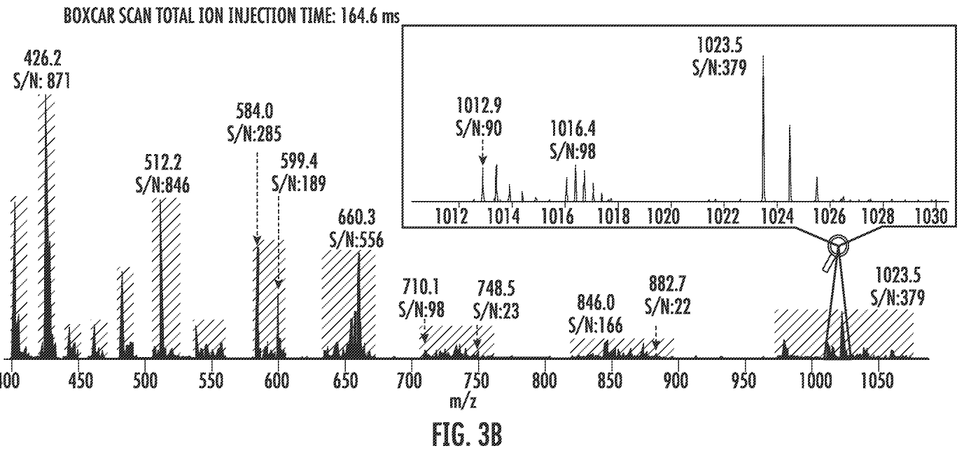

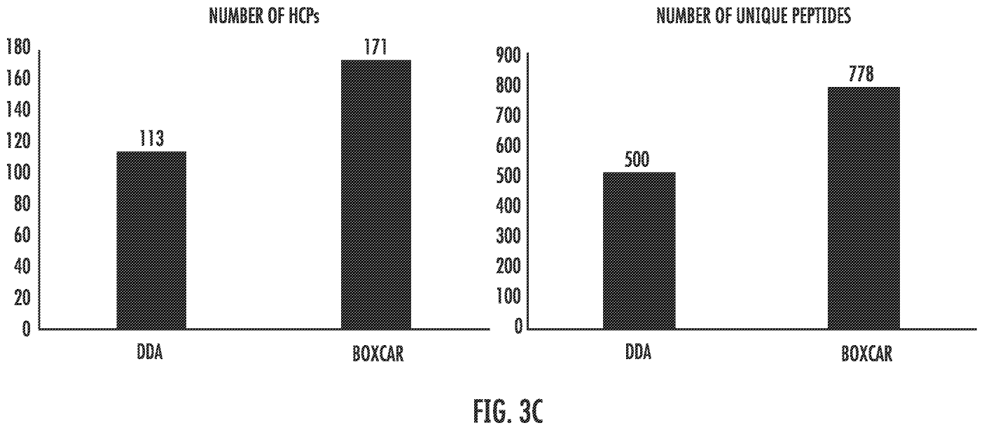

As shown in FIGS. 3A and 3B, the summed ion injection time of the BoxCar scan (164.6 ms) was much longer than the ion injection time of the standard full scan (1.3 ms), and the average ion injection times of individual boxes were more than 10-fold higher than for the standard full scan. The end result was an increase in the full scan signal of low-abundance species with poor signal-to-noise ratios (S/N) by more than 40-fold.

Within one LC-MS experiment, the number of BoxCar scans and the number of boxes per BoxCar scan were fixed. The overall acquisition cycle comprised a standard full scan and two or more BoxCar scans covering a range of m/z 400-1200. More BoxCar scans and more boxes in one scan can increase detection dynamic range, but longer cycle times are needed.

To balance overall cycle time and the number of BoxCar scans, multiple BoxCar settings were investigated. The most HCP identifications were obtained when using one full scan plus two BoxCar scans. Detailed m/z windows for each box in the two BoxCar scans are provided in Table 1.

After optimizing BoxCar settings, BoxCar acquisition was compared to DDA using NISTmAb native digestion samples separated with identical LC conditions (a 2 h gradient on a 25 cm column). 171 HCPs were identified using BoxCar acquisition, which represents about 51% improvement over traditional DDA, while unique peptide identifications increased by about 55% (FIG. 3C).

Accordingly, this example sets out the challenges of HCP detection and sets forth various parameters of illustrative assays that significantly improve the second of three steps of the assay: The Liquid Chromatography Mass Spectrometry (LC-MS) BoxCar Step.

### Example 5

### Assay Design and Results of the Combination Three (3) Step Assay the ULTLB Method

This example describes design aspects of illustrative assays for detecting HCPs in therapeutic antibody preparations by combining all three steps of the assay.

Accordingly, all steps of the above illustrated assays were combined, i.e.: the ultra-low trypsin concentration digestion under non-denaturing conditions, the optimal long LC gradient, and BoxCar acquisition into a new method, coined the “ULTLB method,” which was applied to a standard antibody (NISTmAb) to allow direct comparison with other reports. The ULTLB method identified a total of 453 mouse proteins (for a full list of identified HCPs, see Table 1).

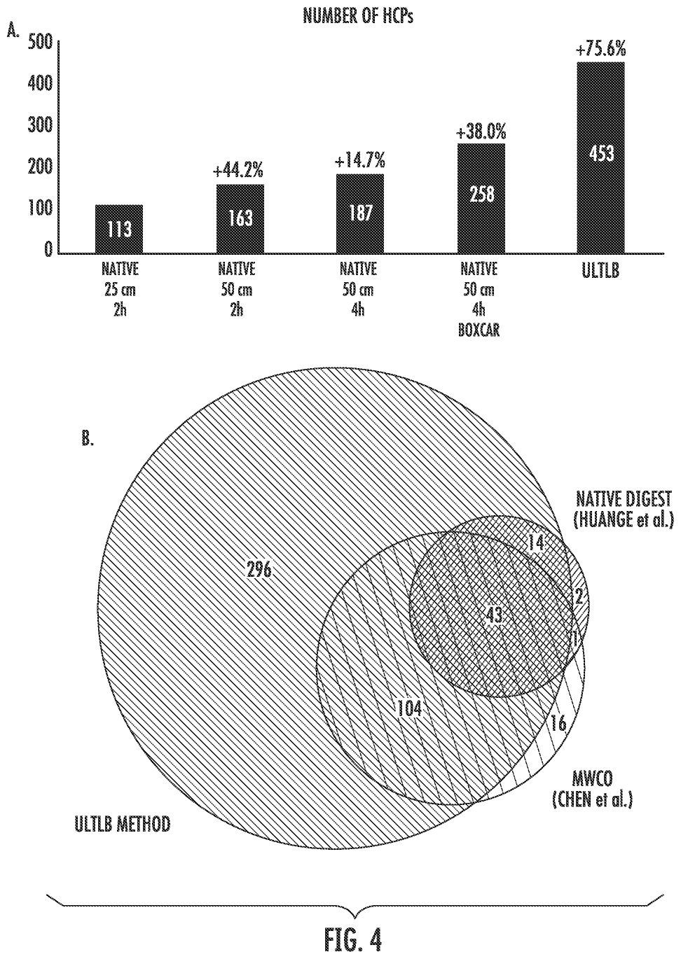

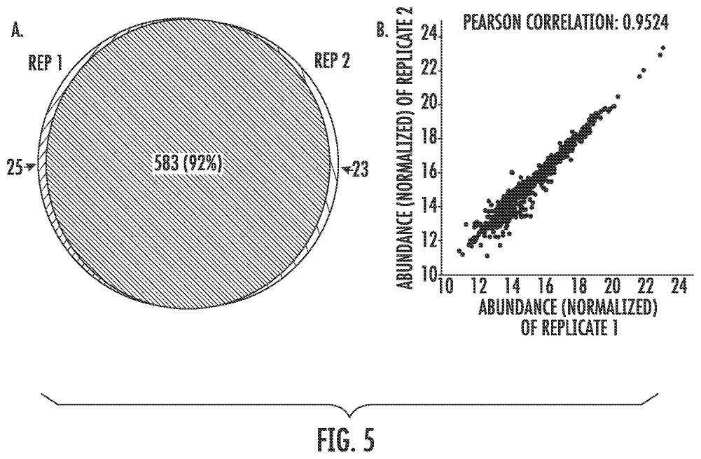

As shown in FIG. 4A, the ULTLB method increased HCP identifications more than four times over the standard native digestion analyzed using a 2 h gradient on a 25 cm column. The results also reveal how much each optimization, including sample preparation, LC separation, and MS acquisition, contributed to improving HCP identifications, where a longer column (50 cm), longer gradient (4 h) and BoxCar acquisition improved HCP identification by 44%, 14% and 38%, respectively. By combining the ultra-low trypsin concentration digestion with optimized LC-MS conditions, HCP identifications were further increased by over 75%.

This indicates that the ultra-low trypsin concentration digestion improved the number of HCP identifications significantly in the ULTLB method. Recently, both Chen et al. (Anal Chem 2020, 92, 3751-3757) and Huang et al. (Anal Chem 2017, 89, 5436-5444) developed HCP identification strategies and tested their efficacy using NISTmAb, identifying 164 HCPs and 60 HCPs, respectively. In addition, 57 of 60 HCPs detected by the native digestion method (Huang et al.) were identified by the ULTLB method and 147 of 164 HCPs identified by the MWCO method were similarly covered by the ULTLB method. In total, 296 mouse HCPs in NISTmAb were identified by the ULTLB method which were not reported in the previous two studies (FIG. 4B).

TABLE 1
Detection limit tested by varying concentrations
of proteins spiked into an antibody sample

| | | | # | # | |
|---|---|---|---|---|---|
| spiked in | | access | identified | unique | MW |
| final ppm | protein name | number | PSM | peptide | [kDa] |
| 200 | Beta- | G3HXN7 | 386 | 21 | 60.1 |
| | hexosaminidase | | | | |
| 100 | complement | G3GUR1 | 566 | 25 | 80 |
| | C1r-A | | | | |
| | subcomponent | | | | |
| 50 | hPLBD2 | G3I6T1 | 332 | 10 | 65.5 |
| 20 | cathepsin Z | Q9EPP7 | 160 | 10 | 34 |
| 10 | sialate O- | G3IIB1 | 32 | 5 | 61.4 |
| | acetylesterase | | | | |
| 10 | cathepsin D | G3I4W7 | 318 | 14 | 44.1 |
| 5 | metallo- | G3IBH0 | 49 | 6 | 22.4 |
| | proteinase | | | | |
| | inhibitor 1 | | | | |
| 5 | peptidyl-prolyl | G3H533 | 203 | 17 | 23.6 |
| | cis-trans | | | | |
| | isomerase | | | | |
| 5 | lysosomal | G3HQY6 | 148 | 8 | 45.6 |
| | acid lipase | | | | |
| 1 | c-x-c motif | A0A061INB9 | 28 | 2 | 39.7 |
| | chemokine | | | | |
| 1 | transtheyretin | G3I4M9 | 40 | 5 | 15.8 |
| 0.5 | acid | G3GZB2 | 17 | 3 | 44.7 |
| | ceramidase | | | | |
| 0.1 | procollagen C | G3I664 | 8 | 1 | 55.2 |
| | endopeptidase | | | | |
| | enhancer 1 | | | | |

To evaluate the detection limit of the method, 13 purified CHO proteins at concentrations ranging from 0.1 to 200 ppm were spiked into a purified commercial-grade monoclonal antibody, mAb1, containing a very low level of endogenous HCPs (Table 1). Important low abundance HCP targets like PLBD2, Cathepsin D, Metalloproteinase inhibitor 1, and others were included in the list. See FIG. 6 for comparative results obtained for two (2) monoclonal antibodies.

The spike-in samples were processed and analyzed in triplicate using the ULTLB method. Table 1 shows that all 13 spike-in proteins were identified with high confidence, and even the lowest concentration protein (0.1 ppm) was identified with eight spectra. Results demonstrate that the ULTLB method is sensitive and can detect HCPs down to 0.1 ppm.

The reproducibility of the ULTLB method was also evaluated across two individual runs using NISTmAb, and a total of 583 proteins, equal to 92% of all proteins, were shared between the runs (FIG. 5A). This reproducible result indicates a high degree of confidence in protein identifications, which is crucial for HCP analysis. Protein abundance was also quantified by label-free quantitation and compared in both runs. The Pearson correlation for this method was greater than 0.95, indicating that the ULTLB method is highly reproducible (FIG. 5B).

Accordingly, this example sets out the challenges of HCP detection and sets forth various parameters and data results of the three step ULTLB method.

### Example 6

### Comparative Analysis of Five (5) Different HCP Analytical Techniques

This example presents a comparative analysis of five (5) different HCP analytical techniques summarized in Table 2.

The present disclosure establishes a simple and novel platform for deep profiling of HCPs, termed the ULTLB method, by combining an ultra-low trypsin concentration during digestion under non-denaturing conditions, a long chromatographic gradient, and BoxCar mass spectrometry acquisition on a quadrupole-Orbitrap mass spectrometer.

Using this strategy, the low abundance HCPs in the mAb samples were preferentially digested using an ultra-low trypsin concentration in non-denaturing conditions, leaving the mAb relatively intact and allowing it to be removed by denaturation or MWCO filter. Optimizing the long gradient separation and BoxCar acquisition further improved the HCP detection dynamic range by 1-2 orders of magnitude.

The spike-in experiments demonstrate the ULTLB method's high sensitivity, achieving a detection limit down to 0.1 ppm. The ULTLB method also detected more than 450 HCPs in NISTmAb with high reproducibility, including almost all identified HCPs previously reported by other methods.

Therefore, the ULTLB method is simple, robust, and enables much deeper profiling of HCPs compared to other methods and obviates additional time-consuming sample enrichment steps.

TABLE 2
Comparison of HCP Assays for Characterizing
the Purity of Therapeutics Antibodies

| | | Extra | | | HCP |
|---|---|---|---|---|---|
| | Time for | Reagents/ | Sample | | IDs in |
| HCP Assay | Sample | Materials | Amount | Sensi- | NIST |
| Methods | Preparation | Needed | (mg) | tivity | mAb |
| IP | +++++ | Antibody | about 10 | about 10 | N/A |
| Pull Down | | Beads | | | |
| Protein A + | +++++ | Protein | about 10 | about 10 | 610 |
| Native | | A Beads | | | |
| Digestion + | | | | | |
| FAIMS | | | | | |
| Native | ++ | W/O | about 10 | about 10 | 60 |
| Digestion | | | | | |
| MW Cutoff | +++++ | 50K | about 10 | about 10 | 165 |
| | | Filter and | | | |
| | | Centrifuge | | | |
| New Method | ++ | W/O | about 10 | about 10 | 453 |

Accordingly, this example sets out an analysis of four (4) preexisting assays for HCP detection as compared to illustrative assays (using ULTB) showing that the ULTLB is superior.

### OTHER EMBODIMENTS

While in the foregoing specification certain embodiments and examples have been described and many details have been put forth for the purpose of illustration, it will be apparent to those skilled in the art that the presently described illustrative assays are susceptible to additional embodiments and that certain details described herein can be varied without departing from the basic principles described herein.

What is claimed is:

1. A method for determining the identity or amount of a contaminating protein in a therapeutic protein sample, comprising:
digesting the protein sample, wherein a smaller polypeptide sequence is obtained;
exposing said digest to a chromatography step; and
exposing the digest of the chromatography step to mass spectrometry (MS), wherein the identity or abundance of the contaminating protein is determined.

2. The method of claim 1, wherein the identity of the contaminating protein is determined by size or sequence.

3. The method of claim 1, wherein the amount of the contaminating protein is determined at a parts per million (ppm) level of less than 10, less than 5, less than 2, less than 1, or less than 0.1.

4. The method of claim 1, wherein the contaminating protein is a host cell protein (HCP).

5. The method of claim 1, wherein the therapeutic protein is an antibody, antibody variant, or antibody fusion.

6. The method of claim 1, wherein the digest is a low trypsin concentration digestion.

7. The method of claim 1, wherein the chromatography is long gradient liquid chromatography.

8. The method of claim 1, wherein the mass spectroscopy (MS) is BoxCar.

9. A method for determining the identity or amount of a contaminating protein host cell protein (HCP) in a therapeutic antibody sample, comprising:
exposing the protein sample to an ultra-low trypsin concentration to produce a protein digest;
exposing said digest to a long gradient liquid chromatography step; and
exposing the digest of the chromatography step to mass spectrometry (MS) BoxCar, wherein the identity or abundance of the contaminating HCP protein is determined.

10. The method of claim 9, wherein the polypeptide is an antibody, antibody variant, or antibody fusion.

11. The method of claim 9, wherein the contaminating protein is selected from the group consisting of Beta-hexosaminidase, complement C1r-A subcomponent, hPLBD2, cathepsin Z, cathepsin D, sialate O-acetylesterase, metalloproteinase inhibitor 1, peptidyl-prolyl cis-trans isomerase, lysosomal acid lipase, c-x-c motif chemokine, transtheyretin, acid ceramidase, and procollagen C endopeptidase enhancer 1.

12. The method of claim 9, wherein the trypsin mediated digest is at a ratio of 10000:1.

13. The method of claim 9, wherein the long gradient liquid chromatography step comprises a 50 cm column and a gradient of 4 hrs.

14. The method of claim 10, wherein the antibody, antibody variant or antibody fusion is selected from the group consisting of aflibercept, rilonacept, alirocumab, dupilumab, sarilumab, cemiplimab, and anti-Ebola antibodies.

15. A polypeptide determined to have a contaminating host cell protein (HCP) at a parts per million (ppm) level of less than 10, less than 5, less than 2, less than 1, or less than 0.1 according to the method of claim 9.

16. The polypeptide of claim 15, wherein the polypeptide is selected from the group consisting of antibody, antibody variant, and antibody fusion.

17. The polypeptide of claim 15, wherein the polypeptide is selected from the group consisting of aflibercept, rilonacept, alirocumab, dupilumab, sarilumab, cemiplimab, and anti-Ebola antibodies.

18. The polypeptide of claim 15, wherein the polypeptide is aflibercept.
